(19) United States
(12) Patent Application Publication
(10) Pub. No.: US 2014/0296721 A1
(43) Pub. Date: Oct. 2, 2014

(54) HEALTH MANAGEMENT DEVICE

(71) Applicant: SEIKO EPSON CORPORATION, TOKYO (JP)

(72) Inventor: Yoshifumi Hano, Suwa (JP)

(73) Assignee: SEIKO EPSON CORPORATION, TOKYO (JP)

(21) Appl. No.: 14/225,846

(22) Filed: Mar. 26, 2014

(30) Foreign Application Priority Data
Mar. 28, 2013 (JP) 2013-068061

(51) Int. Cl. A61B 5/083; A61B 5/024; A61B 5/0205

(52) U.S. Cl. CPC A61B 5/083; A61B 5/0205; A61B 5/024
USPC 600/484; 600/532

(57) ABSTRACT

A health management device includes: a signal output unit which outputs a start signal indicating a start timing when application of an exercise load on a subject is started; an acetone detection unit which detects an acetone concentration in a biogas of the subject; a clock unit which measures time; a storage unit which stores the start signal and the acetone concentration outputted from the acetone detection unit together with the time; and a display unit which displays a first transition data group of the acetone concentration outputted from the acetone detection unit before the start signal is received, and a second transition data group of the acetone concentration outputted from the acetone detection unit after the start signal is received, based on an output from the storage unit.

### BACKGROUND

1. Technical Field

The present invention relates to a health management device or the like which measures acetone contained in a biogas and performs health management accordingly.

2. Related Art

Triglyceride accumulated in internal organs is considered to be a primary factor of metabolic syndrome. In order to reduce the triglyceride, it is known to be effective to continuing aerobic endurance exercises with an appropriate exercise intensity.

The measurement of the amount of activity using a three-dimensional motion sensor and the measurement of pulse rate are carried out for the purpose of grasping the state of exercising. This is aimed at achieving a triglyceride reducing effect by presenting a desirable exercise intensity to the user, using epidemiologic study data on the state of exercising and the neural fat reducing effect. However, there is a problem that some people cannot continue exercising because the preset exercise conditions are difficult for the subjects to continue, or for similar reasons. Changes in the metabolic state resulting from the continued exercising are canceled by quitting the exercise. Therefore, for people who cannot continue exercising, it is difficult to achieve a triglyceride reducing effect.

As a measure to allow the user to actually feel the effect of continued exercising, changes in body composition and body weight are monitored on a daily basis, utilizing an impedance-based body composition meter and body weight scale. However, since these changes are largely influenced by meals and the amount of body water, continuing exercising under recommended conditions does not necessarily lead to intended changes. Consequently, there is a problem that the user's desire to continue exercising falls and therefore the user cannot continue exercising.

As an indicator of the effect of continued exercising, a change in metabolic capacity in an aerobic exercise is known. “Mitochondrial Capacity in Skeletal Muscle Is Not Stimulated by Weight Loss Despite Increases in Insulin Action and Decreases in Intramyocellular Lipid Content,” DIABETES, VOL. 57, April 2008, p. 987, reports that, as a result of comparing a dieting group based solely on dieting and a dieting plus exercising group based on dieting and exercises are compared, the mitochondrial density and the activity of the electron transfer system, which have a large correlation with the aerobic metabolic capacity in aerobic exercises, increase in the latter group.

“The process of change in lipid metabolism-related enzyme activity in skeletal muscle in initial stages of endurance running training,” Journal of Winter Sport, Vol. 7, No. 1, 2004, p. 9-14, reports a study on rats in which the activity of the 3-hydroxyacyl-CoA dehydrogenase (HAD), which is a rate-limiting enzyme of fatty-acid beta oxidation type, and citrate synthase (CS, EC 4.1.3.7) activity, which is an indicator of TCA cycle activity, are measured during a period of continued exercising. It is disclosed that HAD activity significantly increases from the initial period when exercising is started, whereas CS activity significantly increases from three weeks after the start of exercising.

If an indicator that changes according to the period of continued exercising such as aerobic metabolic capacity is measured and shown in a graph so that the person who does the exercise can recognize the effect of the continued exercising, this can lead to continued exercising. However, if the measurement is invasive, the measurement usually requires the attendance of a health care worker and therefore cannot be easily introduced in a training site that is necessary for the maintenance of health. Breath is a biological sample that can be measured non-invasively. In JP-A-2010-268864 and “Review on basic data of variance in breath acetone concentration,” Medical Application of Stable Isotope and Biogas 2, 2010, p. 40-46, the breath acetone concentrations in aerobic exercises are measured.

While JP-A-2010-268864 and “Review on basic data of variance in breath acetone concentration,” Medical Application of Stable Isotope and Biogas 2, 2010, p. 40-46, disclose examples of measurement of breath acetone behavior, for example, if the above data is to be utilized to grasp the effect of continued exercising, it is difficult to grasp the effect of continued exercising simply by comparing the measured data.

### SUMMARY

An advantage of some aspects of the invention is that a health management device is provided that allows the subject to actually feel the effect of continued exercising and thus enhances the subject's motivation, leading to a reduction in fats accumulated in the internal organs as a result of continued exercising.

(1) An aspect of the invention relates to a health management device including: a signal output unit which outputs a start signal indicating a start timing when application of an exercise load on a subject is started; an acetone detection unit which detects an acetone concentration in a biogas of the subject; a clock unit which measures time; a storage unit which stores the start signal and the acetone concentration outputted from the acetone detection unit together with the time; and a display unit which displays a first transition data group of the acetone concentration outputted from the acetone detection unit before the start signal is received, and a second transition data group of the acetone concentration outputted from the acetone detection unit after the start signal is received, based on an output from the storage unit.

According to this configuration, transfer data of the acetone concentration before and after the start of exercise can be displayed. The acetone concentration reflects the metabolic state before the start of the exercise and change in metabolic behavior induced during the exercise and therefore shows complex changes in the concentration. The acetone concentration is particularly influenced by the glycogen concentration in the muscles, the amount of ATP that is necessary for the maintenance of health, the amount of free fatty acids (FFA) supplied from triglyceride (glycerol+fatty acids), the blood free fatty acid (FFA) concentration, and the blood glucose concentration. Therefore, time transition of the acetone concentration contained in the biogas is considered to reflect a significant part of the aerobic metabolic behavior during the exercise that is done. Since the metabolic behavior changes largely before and after the exercise, behavior analysis needs to be carried out separately before the start of the exercise and during the exercise, by correctly recognizing the start of the exercise. Also, since changes in the acetone concentration during the exercise show different behaviors according to the exercise intensity, the change in concentration during the exercise may not necessarily be greater than the change in concentration when the subject is resting before the start of the exercise. Even in such a case, the difference between the metabolic state before the start of the exercise and the metabolic state during the exercise can be estimated by grasping the start point of the exercise.

(2) In one aspect of the invention, the health management device may be configured such that the signal output further outputs an end signal indicating an end timing when the application of the exercise load on the subject is ended, the storage unit further stores the end signal and the acetone concentration outputted from the acetone detection unit together with the time, and the display unit displays a third transition data group of the acetone concentration outputted from the acetone detection unit after the end signal is received, together with the first transition data group and the second transition data group.

Since the metabolic behavior changes before the start of the exercise, during the exercise and after the end of the exercise, behavior analysis needs to be carried out separately before the start of the exercise, during the exercise and after the end of the exercise, by correctly recognizing the start of the exercise and the end of the exercise. For example, if the exercise intensity is high or the duration of the exercise is long, lactic acid may be accumulated in the muscles. As the exercise is suspended, glucose generated in the liver through gluconeogenesis using lactic acid or the like is supplied to the muscles and the muscle glycogen consumed during the exercise is regenerated. As a result of an examination, the inventor considers that since the concentration of blood acetoacetic acid generated in the gluconeogenesis and supplied from the liver is high, the majority of the acetoacetic acid is metabolized as acetone. That is, if exercise using a large amount of blood glucose and muscle glycogen as an energy supply substrate is carried out, a rise in the acetone concentration after the end of the exercise can be confirmed through the display.

(3) In one aspect of the invention, the health management device may be configured such that the health management device further includes an analysis unit which analyzes a metabolic state, based on at least one of the first transition data group, the second transition data group and the third transition data group stored in the storage unit, and the display unit displays a result of analysis from the analysis unit.

The acetone concentration reflects the metabolic state before the start of the exercise and change in metabolic behavior induced during the exercise and therefore shows complex changes in the concentration. The acetone concentration is particularly influenced by the glycogen concentration in the muscles, the amount of ATP that is necessary for the maintenance of exercise and generated in the TCA cycle, the amount of free fatty acids (FFA) supplied from triglyceride (glycerol+fatty acids), the blood free fatty acid (FFA) concentration, and the blood glucose concentration. Therefore, time transition of the acetone concentration contained in the biogas is considered to reflect a significant part of the aerobic metabolic behavior during the exercise that is done. Since the metabolic behavior changes largely before the exercise, during the exercise and after the exercise, behavior analysis needs to be carried out separately before the start of the exercise, during the exercise and after the exercise, by correctly recognizing the start of the exercise and the end of the exercise.

(4) In one aspect of the invention, the health management device may be configured such that the health management device further includes a recording unit which records meal time, and the analysis unit analyzes the metabolic state including a correlation with the meal time, based on at least one of the first transition data group, the second transition data group and the third transition data group stored in the storage unit.

The energy substrate used during the exercise depends not only on the exercise intensity and the duration of the exercise but also on the blood glucose level. Since the blood glucose level at the start of the exercise is different between exercise shortly after a meal and exercise during a fasting time, the behavior is totally different with the same intensity and duration of the exercise. As energy demand during the resting time before the start of the exercise is relatively stable, time transition of the acetone concentration during the resting time show a behavior reflecting the glucose concentration and the state of liver glycogen. When a diabetic patient does an aerobic exercise, if glucose is overused by the exercise, there is a risk of hypoglycemia particularly after the exercise. Although it is difficult to predict the blood glucose level before the exercise by using an activity meter or by measuring a pulse rate, constant monitoring of time transition of the acetone behavior enables prediction of such risks without any invasive measurement.

Meanwhile, if a person does an aerobic exercise for the purpose of enhancement of health such as improvement in metabolic capacity, a moderate fasting time when the acetone concentration is stable or gently rises in the case of resting, can be analyzed and recommended as a suitable exercising timing.

(5) In one aspect of the invention, the health management device may be configured such that the signal output unit is a biological signal detection unit which detects a biological signal that changes according to the magnitude of the exercise load on the subject, the biological signal is stored in the storage unit, and the display unit displays the biological signal.

The signal output unit may be, for example, a switch operated by the subject, an acceleration sensor or a gyro sensor installed on the subject, or a drive unit or the like of an exercise machine which outputs a start/end of operation. However, the signal output unit may also be the biological signal detection unit which detects the biological signal that changes according to the magnitude of the exercise load on the subject. The biological signal of this type may be a pulse rate, heart rate or the like that can be measured non-invasively.

(6) In one aspect of the invention, the health management device may be configured such that the biological signal detection unit is a pulse rate detection unit which detects a pulse rate of the subject. The pulse rate can be measured non-invasively at the fingertip, on the wrist or the like, and change according to the magnitude of the exercise load on the subject. If the load is larger, the pulse rate is higher. If the load is smaller, the pulse rate is lower. The pulse rate can be used as a start signal and end signal.

(7) In one aspect of the invention, the health management device may be configured such that the recording unit is an ethanol detection unit which detects an ethanol concentration in the biogas of the subject. The ethanol concentration in the biogas rises and reaches a maximum value after a meal, and then gently lowers. Therefore, the meal time can be found from the clock unit, using the time when the ethanol concentration rises as reference time.

(8) In one aspect of the invention, the health management device may be configured such that data groups about the same subject are accumulated repeatedly in the storage unit, the analysis unit analyzes an exercise level that is recommended with respect to at least one of the exercise intensity and the duration of the exercise, based on the data groups accumulated in the storage unit, and the display unit displays the recommended exercise level from the analysis unit.

The acetone concentration usually changes during exercise. However, if there is no change in the acetone concentration, it is the case where the subject has a high metabolic capacity for fatty acid during a resting time (before exercise) and where the metabolic capacity for fatty acid and the glucose metabolic capacity during the resting time can provide the necessary energy at the implemented exercise level. Therefore, if a person does an aerobic exercise for the purpose of improvement of metabolic syndrome and enhancement of health such as improvement in metabolic capacity, an instruction to raise the exercise level can be given.

(9) In one aspect of the invention, the health management device may be configured such that if the amount of the data groups that are accumulated is small, the analysis unit recommends an exercise menu selected from plural initial exercise menus (protocols, recipes) in which at least one of the exercise intensity and the duration of the exercise varies, based on the data groups. Even if the accumulated data is small, an initial exercise menu can be recommended, based on the measured data groups, and an effective aerobic exercise can be designated.

### BRIEF DESCRIPTION OF THE DRAWINGS

The invention will be described with reference to the accompanying drawings, wherein like numbers reference like elements.

FIG. 1 is a block diagram of a control system of a health management device according to a first embodiment of the invention.

FIG. 2 shows a model example in the case where acetone concentration due to exercising is measured.

FIG. 3 shows a fat burning mechanism in exercising.

FIG. 4 shows an example of the pattern of the result of analysis based on acetone concentration detected before exercise.

FIG. 5 shows an example of the display of the result of detection for a subject 1.

FIG. 6 shows an example of the display of the result of detection for a subject 2.

FIG. 7 shows an example of the display of the result of detection for a subject 3.

FIG. 8 shows an example of the display of the result of detection for a subject 4.

FIG. 9 shows an example of the display of the result of detection for a subject 5.

FIG. 10 is a flowchart showing the former half of an operation flow.

FIG. 11 is a flowchart showing the latter half of the operation flow.

FIG. 12 shows an example of an initial exercise menu.

FIG. 13 is a graph showing the concentration of acetone and ethanol in skin gas in terms of each site where the gas is generated.

FIG. 14 shows the state of use of a skin gas detection device.

FIG. 15 is a front view of the skin gas detection device.

FIG. 16 is a cross-sectional view of the skin gas detection device.

FIG. 17 shows the flow of air in the skin gas detection device.

FIG. 18 is a timing chart showing the operation timing of a sample suction unit and a detection unit.

FIGS. 19A and 19B show a semiconductor sensor.

FIG. 20 shows the relation between the resistance value of the semiconductor sensor and the gas concentration.

FIGS. 21A and 21B show an example of a QCM sensor.

FIGS. 22A and 22B show another example of a QCM sensor.

FIG. 23 shows calibration curves between the frequency change rate of the QCM sensor and the gas concentration.

FIG. 24 illustrates the detection principle of a SERS sensor.

FIGS. 25A and 25B show a pad-type skin gas detection device according to a second embodiment of the invention.

FIGS. 26A and 26B are a front view and cross-sectional view of the pad-type skin gas detection device.

FIG. 27 shows an exercise machine in which the skin gas detection device according to the second embodiment of the invention is incorporated.

FIG. 28 shows a hand portion shown in FIG. 27.

FIG. 29 is a cross-sectional view showing a grip portion of the hand shown in FIG. 27.

### DESCRIPTION OF EXEMPLARY EMBODIMENTS

Hereinafter, preferred embodiments of the invention will be described in detail. The embodiments described below are not to unduly limit the content of the invention. Not all the configurations described in the embodiments are essential as the means for solution according to the invention.

### 1. First Embodiment

### 1.1. Health Management Device

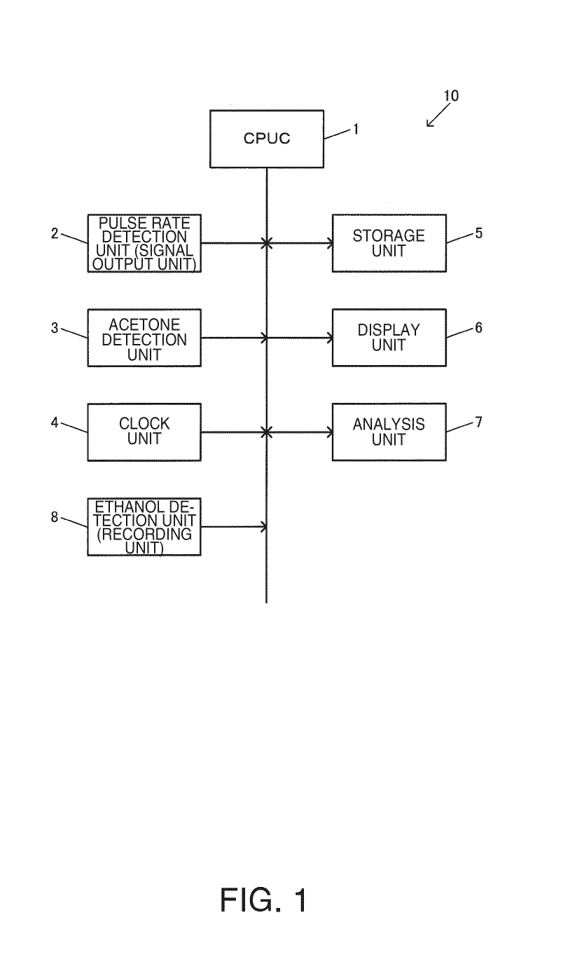

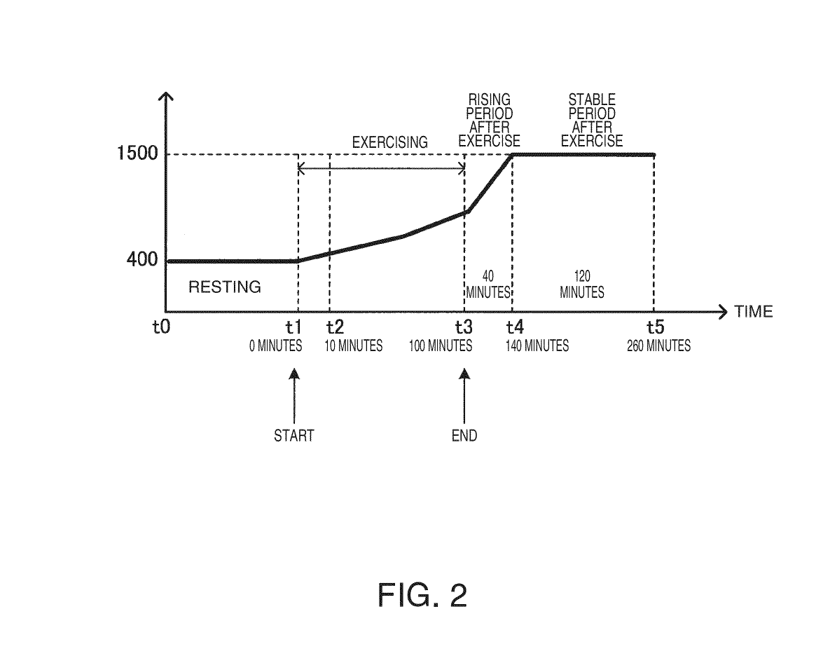

FIG. 1 is a block diagram of a control system of a health management device 10 according this embodiment. FIG. 2 shows a model example of acetone concentration transition data displayed on a display unit. In FIG. 1, the health management device 10 includes a pulse rate detection unit (in a broad sense, a signal output unit) 2, an acetone detection unit 3, a clock unit 4, a storage unit 5, and a display unit 6, provided on a bus line of a CPU 1 as a main controller responsible for overall control. An analysis unit 7 and an ethanol detection unit (in a broad sense, a recording unit) 8 can also be provided on the bus line of the CPU 1.

The pulse rate detection unit 2 detects the pulse rate, which is a biological signal of the subject. The pulse rate can be recognized, for example, optically, at the fingertip or on the wrist of the subject. The pulse rate detection unit 2 is an example of the signal output unit and is configured to output at least a start signal indicating a start timing when application of an exercise load on the subject is started. The pulse rate rises as the application of the exercise load on the subject is started and exercise is started. Therefore, the start timing can be extracted from the pulse rate. The signal output unit 2 can output an end signal indicating an end timing when the application of the exercise load on the subject is ended. The pulse rate falls as the application of the exercise load on the subject is ended and the exercise is ended. Therefore, the end timing can be extracted from the pulse rate.

The acetone detection unit 3 detects the acetone concentration in the subject's biogas. The acetone detection unit 3, the detail of which will be described later, can detect acetone in the subject's breath or skin gas. The clock unit 4 measures time. The storage unit 5 stores at least the start signal and the acetone concentration outputted from the acetone detection unit 3 in a period before or after the reception of the start signal together with time. The storage unit 5 can also store the end signal and the acetone concentration outputted from the acetone detection unit 3 during a period after the reception of the end signal together with time. The period before or after the reception of the start signal is between a necessary period for measurement and analysis as a minimum period and a period that can be implemented with the capacity of the storage medium as a maximum period.

FIG. 2 shows an example of the display on the display unit 6. In FIG. 2, the horizontal axis represents time elapsed and the vertical axis represents acetone concentration (ppb). The display unit 6 displays a first transition data group of the acetone concentration outputted from the acetone detection unit 3 before the reception of the exercise start signal (t0 to t1) and a second transition data group of the acetone concentration outputted from the acetone detection unit 3 after the reception of the exercise start signal and before the reception of the exercise end signal (t1 to t3), based on an output from the storage unit 5. In FIG. 2, an example in which the time of reception of the exercise start signal is shown as 0 minutes and then the exercise end signal is received at 100 minutes, thus amounting to 100 minutes of exercise, is shown. In the example of FIG. 2, the display unit 6 can also display a third transition data group of the acetone concentration outputted from the acetone detection unit 3 after the reception of the exercise end signal (after t3).

The analysis unit 7 analyses the metabolic state, based on at least one of the first transition data group, the second transition data group and the third transition data group stored in the storage unit 5. The result of this analysis can be displayed on the display unit 6. The ethanol detection unit 8 detects ethanol in the subject's breath or skin gas. The ethanol detection unit 8 is an example of a recording unit which records meal time. The ethanol concentration in the biogas shows a unique pattern in which the ethanol concentration rises and reaches a maximum value after a meal, and then gently falls. Therefore, the meal time may be found from the clock unit 4, using the time when the ethanol behavior is observed as reference time. In this case, the analysis unit 7 can analyze the metabolic state including the correlation with the meal time, based on at least one of the first transition data group, the second transition data group and the third data transition group stored in the storage unit 5. These analysis examples will be described below.

### 1.2. Analysis Based on Acetone Concentration Transition Data

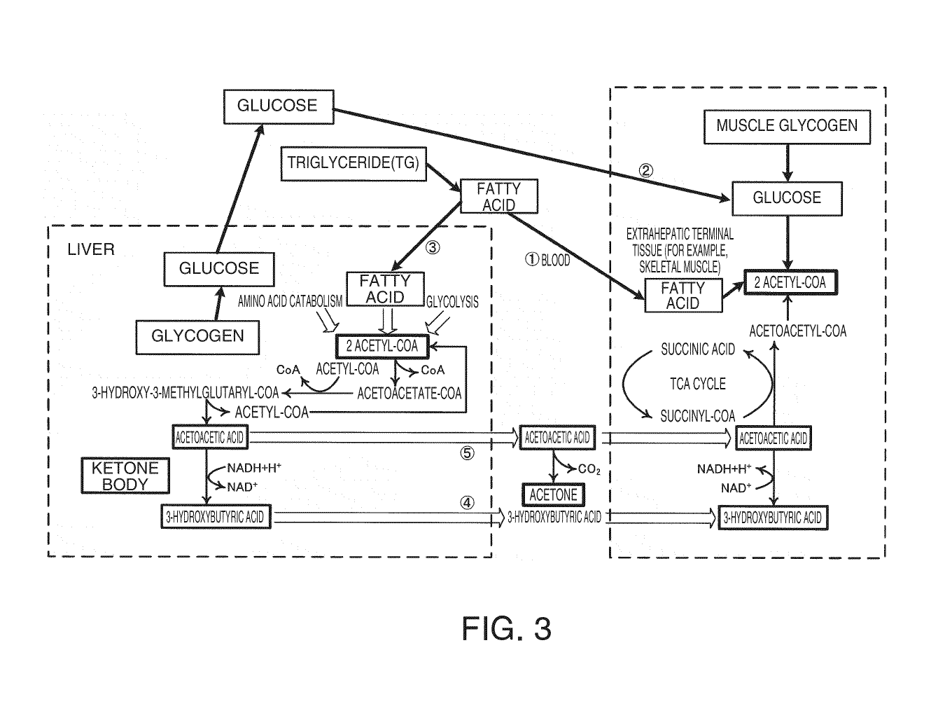

FIG. 3 (modification of Lippincott's Illustrated Biochemistry P.247) shows a simplified example of a fat burning mechanism in exercising. The acetone concentration reflects the metabolic state before the start of the exercise and change in metabolic behavior induced during the exercise and therefore shows complex changes in the concentration. The acetone concentration is particularly influenced by the glycogen concentration in the muscles, the amount of ATP that is necessary for the maintenance of exercise and generated in the TCA cycle, the amount of free fatty acids (FFA) supplied from triglyceride (glycerol+fatty acids), the blood free fatty acid (FFA) concentration, and the blood glucose concentration. Therefore, time transition of the acetone concentration contained in the biogas is considered to reflect a significant part of the aerobic metabolic behavior during the exercise that is done. Since the metabolic behavior changes largely before the exercise, during the exercise and after the exercise, behavior analysis needs to be carried out separately before the start of the exercise, during the exercise and after the exercise, by correctly recognizing the start of the exercise and the end of the exercise.

### 1.2.1. Example of Analysis Based on Acetone Concentration Transition Data Before Start of Exercise

Since energy demand during the resting time before the start of the exercise is relatively stable, time transition of the acetone concentration during the resting time show a behavior reflecting the resting metabolic state, the blood glucose concentration and the amount of accumulated liver glycogen.

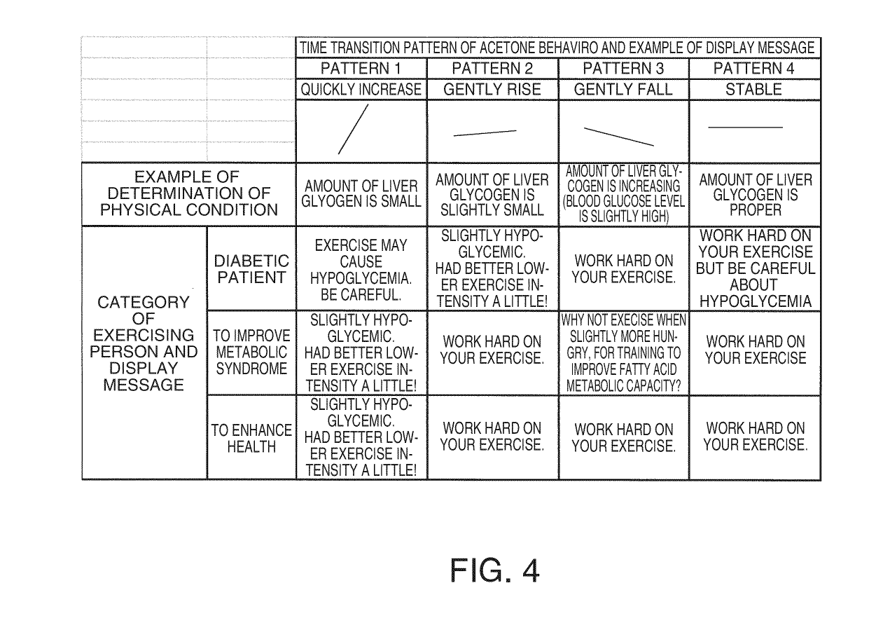

FIG. 4 shows patterns 1 to 4 that are results of analysis by the analysis unit 7 shown in FIG. 1, based on acetone concentration transition data before the start of exercise. For example, if a diabetic patient does exercise in the state where the acetone concentration quickly increases (pattern 1) or gently rises (pattern 2) before the start of the exercise, and consumers too much glucose through the exercise, there is a risk of hypoglycemia. Thus, if the analysis unit 7 analyses that the present pattern is the pattern 1 or 2, the message shown in FIG. 4 can be displayed on the display unit 6 as the result of the analysis. Although it is difficult to predict the blood glucose level before the exercise by using an activity meter or by measuring the pulse rate, constant monitoring of time transition of the acetone behavior enables prediction of such risks without any invasive measurement.

In contrast to the patterns 1 and 2, the acetone concentration may gently fall (pattern 3). Before a fall in the blood glucose level that is raised after a meal, the acetone concentration gently falls even when resting. If the analysis unit 7 analyzes that the present pattern is the pattern 3, the message shown in FIG. 4 can be displayed on the display unit 6 as the result of the analysis. For the pattern 3, the ethanol concentration transition detected by the ethanol detection unit 8 also serves as a criterion for determination.

At the time of moderate fasting of the pattern 4, if the blood glucose level is back to the normal level, the acetone concentration is stable and has little change (see FIG. 2). Therefore, there is no particular need to display a warning message on the display unit 6. Specific examples of the patterns 1 to 4 will be described later.

### 1.2.2. Example of Analysis Based on Acetone Concentration Transition Data During of Exercise

In an exercise using fast twitch muscles where a heavy load acts in a short time, glucose is consumed (route 2 in FIG. 3). On the other hand, in an endurance exercise (aerobic exercise) of medium or lower intensity using slow twitch muscles, fatty acids are mainly used while restraining the consumption of the finite glucose. Exercising promotes decomposition of triglyceride, which turns into fatty acids and glycerol. Since fatty acids cannot circulate through the blood as they are, fatty acids are bound with albumin to form free fatty acids (FFAs), which then circulate through the blood.

The capacity to directly take fatty acids into mitochondria in the muscles (route 1 in FIG. 3) is limited. A predetermined amount of fatty acids is decomposed in the liver (route 3 in FIG. 3) and supplied in the form of ketone bodies generated from acetyl-CoA. The “ketone bodies” is a general term for acetoacetic acid, beta-hyhdroxybutyric acid (3-hydroxybutyric acid), and acetone. Acetoacetic acid is generated from acetyl-CoA. Acetoacetic acid exists in equilibrium with beta-hydrocybutyric acid. The equilibrium changed according to the NADH concentration in liver mitochondria.

Acetoacetic acid and beta-hydroxybutyric acid are discharged into blood as they are and carried to the terminal tissues (routes 4 and 5 in FIG. 3). In the terminal tissues, acetoacetic acid and beta-hydroxybutyric acid are used to re-synthesize acetyl-CoA with succinyl-CoA generated by the TCA cycle in mitochondria of the terminal tissues.

When acetoacetic acid moves through the blood, apart of the acetoacetic acid is decomposed into acetone. Acetone is not used in the body and diffused outside the body by gas exchange through the lungs and skin. Particularly when the blood acetoacetic acid concentration and beta-hydroxybutyric acid concentration are high, a significant part of the acetoacetic acid is metabolized as acetone.

### 1.2.3. Example of Analysis Based on Acetone Concentration After End of Exercise

Arise in the acetone concentration is also observed after exercise as well as during exercise. During the endurance exercise as described above, free fatty acids (FFAs) are used as a main substrate. However, if the exercise intensity is high or the duration of the exercise is long, in the latter part of the exercise, the proportion of glucose that is used increases and anaerobic glycolysis of glucose takes place and therefore lactic acid is accumulated in the muscles. As the exercise is suspended, glucose is regenerated through gluconeogenesis using lactic acid and amino acids and used for regeneration of muscle glycogen that is consumed when the exercise is started.

In this embodiment, it is found that the degree of rise in the acetone concentration after exercise is correlated with the aerobic exercise capacity of the exercising person and the state of exercising that is implemented. Therefore, it is found that the rise in the acetone concentration after exercise is likely due to acetoacetic acid generated through the gluconeogenesis in the liver. That is why the acetone concentration rises shortly after the suspension of exercise, as shown in FIG. 3.

Also, depending on the circumstance, the acetone concentration may rise further, following the rise in the acetone concentration shortly after exercise. After the amount of muscle glycogen is recovered to a certain level, a metabolic state similar to that of the resting time is recovered if the amount of liver glycogen is large enough. However, if a large amount of liver glycogen is consumed in aerobic exercise, it is difficult to maintain a necessary blood glucose level. Thus, it can also be considered that the behavior of supplying a part of the energy for maintaining the resting state, as ketone bodies, is observed as the foregoing behavior.

Therefore, since the acetone behavior after the suspension of exercise is considered to reflect the amount of muscle glycogen consumed and the amount of blood glucose consumed in aerobic exercise, and in the case of fasting, the amount of liver glycogen consumed, it can be considered that separate analysis of each acetone behavior after the suspension of exercise can provide useful information about energy consumption behavior in aerobic exercise.

### 1.3. Specific Example of Acetone Concentration Transition Data

### 1.3.1 Subject 1

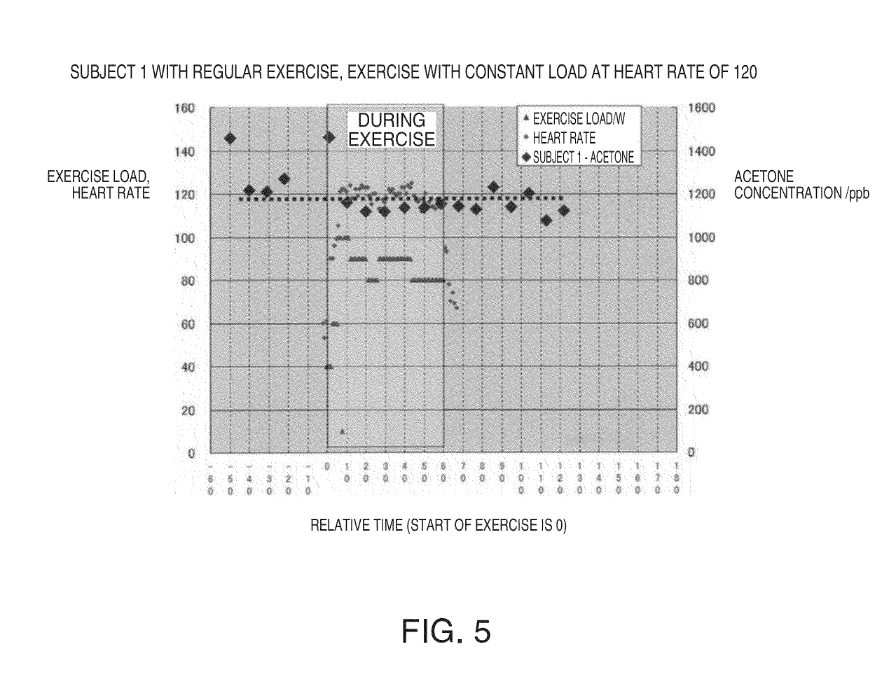

The result of acetone concentration measurement in the case where a subject 1 carries out an endurance exercise for 55 minutes at a heart rate of 120 when fasting three hours after a meal is shown in FIG. 5. With this subject 1, little change is observed in the trend of acetone concentration change before the exercise, during the exercise and after the exercise. Since the metabolic state changes due to various factors even during the resting time, it is desirable to ignore variations in the acetone concentration particular during the resting time, and grasp the acetone behavior as a trend. Thus, the analysis unit 7 can analyze that the acetone concentration transition data before the exercise matches the pattern 4 in FIG. 4 (moderate fasting state). The analysis unit 7 also analyzes that the aerobic metabolic capacity during the exercise with respect to the exercise load of this time and the aerobic metabolic capacity of the muscles at the start of the exercise with respect to the exercise load of this time are both ranked at 10.

That fact that there is little change in the acetone concentration before the exercise, during the exercise and after the exercise can be analyzed as indicating that the subject 1 has a high aerobic metabolic capacity for fatty acid and glucose when resting. It means that the subject's high aerobic metabolic capacity for fatty acid and glucose of the resting time can even cover the endurance exercise of 55 minutes at the heart rate of 120 with. Also, the fact that there is no rise in the acetone concentration after the exercise can be interpreted as indicating that the accumulation of lactic acid is small and therefore gluconeogenesis has no influence on the rise in the acetone concentration. Therefore, in addition to the determination that the present pattern is the pattern 4 of FIG. 4, the analysis unit 7 can analyze that it is desirable to carry out the endurance exercise with a slightly heavier exercise load for improvement in metabolic function.

### 1.3.2. Subject 2

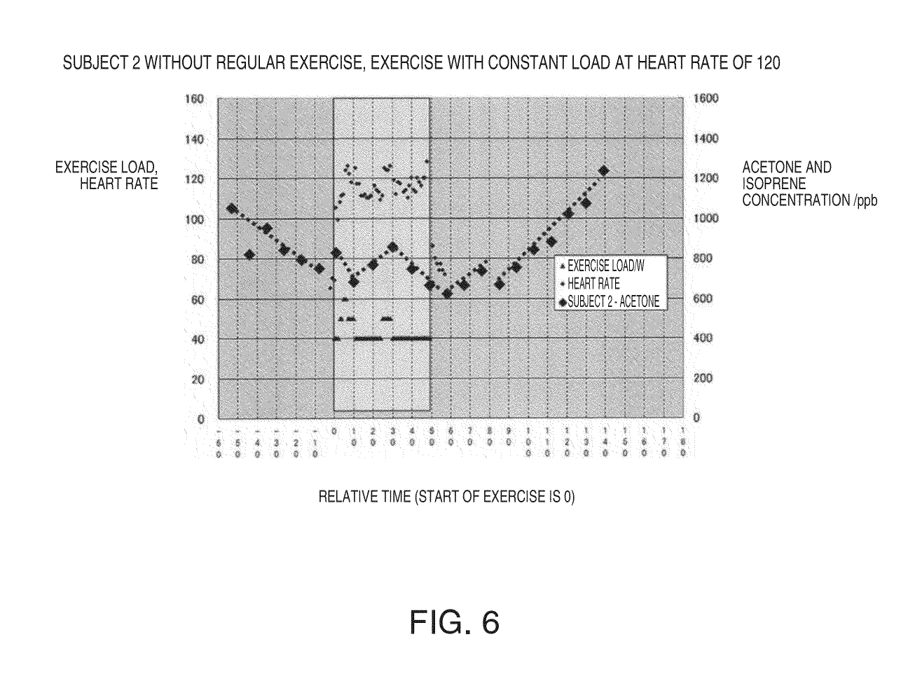

The result of acetone concentration measurement in the case where another subject 2 carries out an endurance exercise for 45 minutes at a heart rate of 120 when fasting is shown in FIG. 6. With this subject 2, (1) a fall in the acetone concentration before the exercise, (2) a sudden fall in the acetone concentration 10 minutes after the exercise, (3) a quick rise in the acetone concentration after that, (4) a sudden fall in the acetone concentration 30 minutes after the start of the exercise, and (5) two stages of quick rise in the acetone concentration after the suspension of the exercise are observed.

Based on the behavior (1), the state before the exercise can be considered to be a stage some time after a quick rise in the blood glucose level following a meal (pattern 3 in FIG. 4). Glucose metabolism during the exercise is considered dominant. However, since the behavior (5) is observed, which is regarded as a rise in the acetone concentration due to gluconeogenesis, the exercise intensity implemented this time is considered high for the subject 2. Therefore, it is recognized that the aerobic metabolic capacity with respect to the exercise load of this time is ranked at 3.

Moreover, based on the behaviors (2) to (4), it can be determined that, after the lapse of 30 minutes from the start of the exercise, the actual fatty acid metabolic capacity of the subject 2 is below the necessary fatty acid metabolic capacity to maintain the exercise, while the proportion of glucose metabolism is increased. Therefore, it is recognized that the aerobic metabolic capacity during the exercise with the respect to the exercise load of this time is ranked at 2.

Judging from these behaviors, the exercise intensity and the amount of exercise of this time are too high for the subject 2 and the subject 2 is likely to be fatigued. In such a case, the fatigue can remain the following day, and even if the subject recovers from the fatigue, the subject is likely to have a lower desire to exercise and therefore give up continuing the exercise. Thus, by proposing an exercise with significantly lower exercise intensity (for example, at a heart rate of approximately 100) for the next exercise session, the amount of exercise can be suitable for the current metabolic capacity.

By adjusting the exercise load according to the metabolic capacity, the subject can complete the preset exercise and excessive fatigue accumulation does not take place. Therefore, this adjustment is a desirable measure for the purpose of continued exercising. Even in this case, if the subject continues exercising, improvement in the metabolic capacity corresponding to the exercise that is done can be achieved and the subject can feel an actual effect of the continued exercising. Then, if the subject carries out the exercise implemented this time at the stage where the metabolic capacity is improved, the subject is likely to be able to complete the exercise and experience significantly less fatigue, in contrast to this time.

Generally, if the subject has a low aerobic metabolic capacity, the acetone concentration when resting tends to be low. However, the resting acetone concentration before the exercise of this time is high. This can be due to the insufficient amount of glucose supplemented by the meal due to dieting or the like, and as a result of exercising, the shortage of glucose as a substrate, instead of the metabolic capacity, can be the cause of the acetone behavior of this time. Thus, in the determination, it is important to store acetone concentration transition information during non-exercising time and notify that alimentation is not sufficient if that is the case.

### 1.3.3. Subject 3

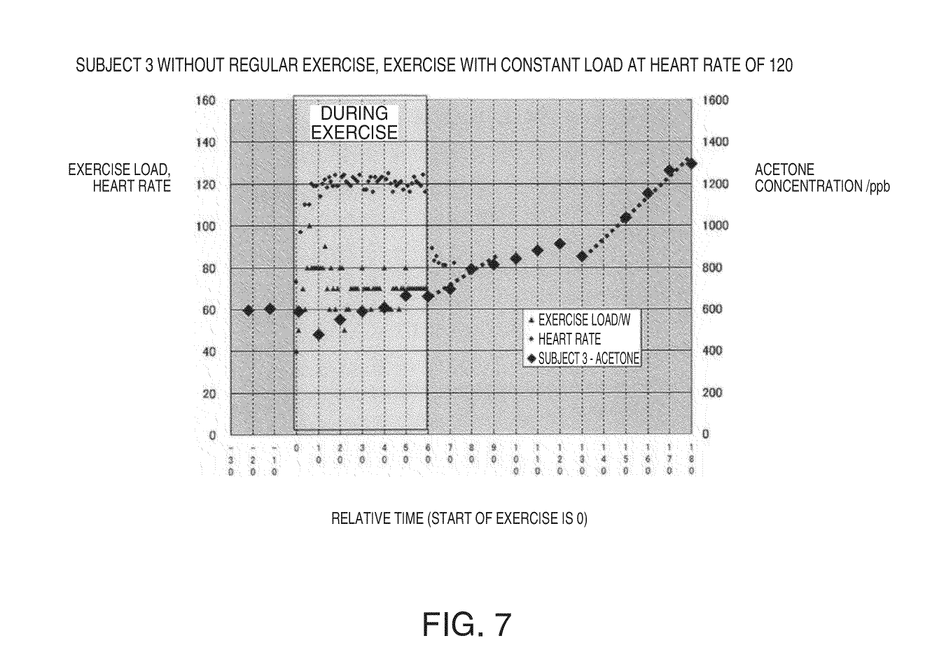

The result of acetone concentration measurement in the case where another subject 3 carries out an endurance exercise for 55 minutes at a heart rate of 120 when fasting is shown in FIG. 7. The acetone concentration before the start of the exercise is stable and recognized as the pattern 4 in FIG. 4 (moderate fasting). The acetone concentration falls during 10 minutes after the start of the exercise and then gradually rises. Therefore, it is recognized that the aerobic metabolic capacity during the exercise with respect to the exercise load of this time is ranked at 5, which is the average level. Moreover, since the acetone concentration does not quickly rise shortly after the end of the exercise, it is recognized that the aerobic metabolic capacity of the muscles at the start of the exercise with respect to the exercise load of this time is ranked at 6, which is higher than the average level. Following the rise shortly after the end of the exercise, the acetone concentration temporarily relaxes and then quickly rises again and continues rising. This is considered to reflect the shortage of the amount of liver glycogen as a result of large consumption of blood glucose during the exercise. Therefore, it is recognized that if the subject carries out the exercise of this time, the subject can become hypoglycemic after the exercise, for example three hours after the start of the exercise or later.

### 1.3.4. Subject 4

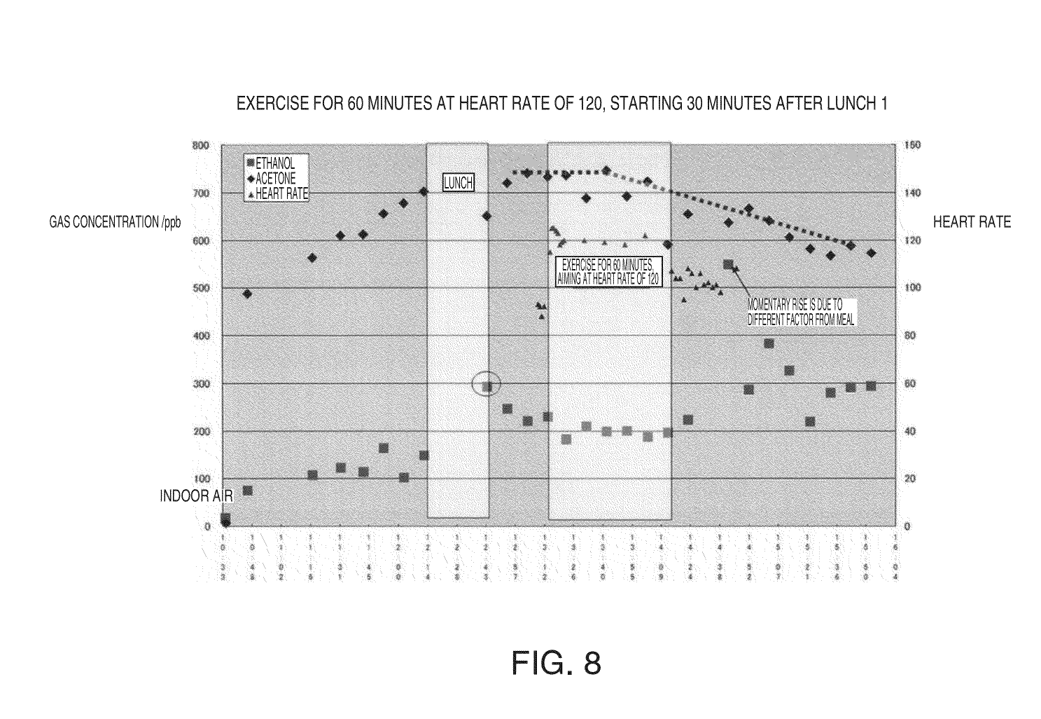

The result of acetone and ethanol concentration measurement in the case where still another subject 4 carries out an endurance exercise for 55 minutes at a heart rate of 120, starting 30 minutes after a meal, is shown in FIG. 8. It is known that, after a person has a meal, the postprandial ethanol concentration shows a behavior of rising and reaching a maximum value and then gently falling. The change in the ethanol concentration is said to be dependent on the content of the saccharide ingested, for example, whether it is monosaccharide or polysaccharide, and the degree of digestion or the like. The time when the ethanol concentration reaches the maximum value is immediately after the meal, in the earliest case. Even in the latest case, the ethanol concentration tends to reach the maximum value approximately 30 minutes after the meal.

Since the blood glucose concentration is high after the meal, if no exercise is done, excess glucose is metabolized into fatty acids and accumulated as triglyceride. If aerobic exercise is carried out, the excess glucose is consumed as an energy source to maintain the exercise, and the blood glucose concentration falls, too. With a diabetic subject, it is considered desirable to carryout aerobic exercise after a meal in order quickly lower the blood glucose level after the meal. Such exercise is adopted as an exercise therapy.

Meanwhile, in the case of a subject who wants to improve the metabolic state, since the glucose that is accumulated as triglyceride when no exercise is done is consumed by exercising, it is desirable to carry out aerobic exercise even after lunch. However, the amount of fatty acids consumed is different from the fatty acid consumption during fasting. Therefore, the change in the acetone concentration due to fatty acids shows a significantly different behavior from that of the fasting time.

Thus, if data in the case where aerobic exercise is carried out when fasting and data in the case where aerobic exercise is carried out after a meal are similarly analyzed and converted into indicators, it is expected that changes in the aerobic metabolic capacity that is improved by continued aerobic exercise cannot be reflected correctly. Since it is difficult to record each meal time, fasting time can be automatically calculated, using indicators that change in behavior because of the meal, such as the ethanol concentration, and indicators that change in behavior because of the exercise, such as the amount of activity and heart rate (pulse rate), and the state before the exercise can be estimated. Thus, the metabolic capacities can be compared in the same state.

### 1.3.5. Subject 5

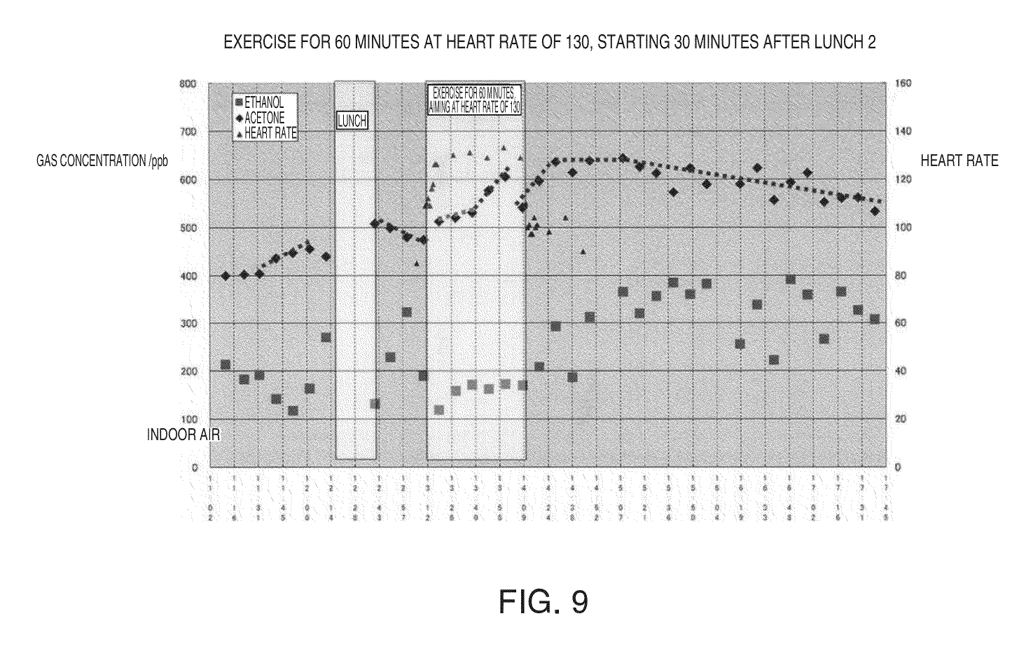

The result of acetone and ethanol concentration measurement in the case where still another subject 5 carries out an endurance exercise for 55 minutes at a heart rate of 130, starting 30 minutes after a meal, is shown in FIG. 9. Although fatty acid metabolism usually does not increased during exercise, fatty acids are mobilized here to deal with high energy that is necessary to maintain the exercise. In the case of a subject who wants to improve the metabolic state, since fatty acids are consumed less in aerobic exercise after lunch than when fasting, it is better to set a higher exercise intensity than usual in order to consume fatty acids. Since the glucose concentration is high, a moderate increase in the exercise intensity is expected to have few adverse effects such as fatigue. If a high exercise intensity is designated in the case of exercise after a meal, improvement in physical strength and consumption of fatty acids can be realized without causing fatigue.

### 1.4. Operation Flow

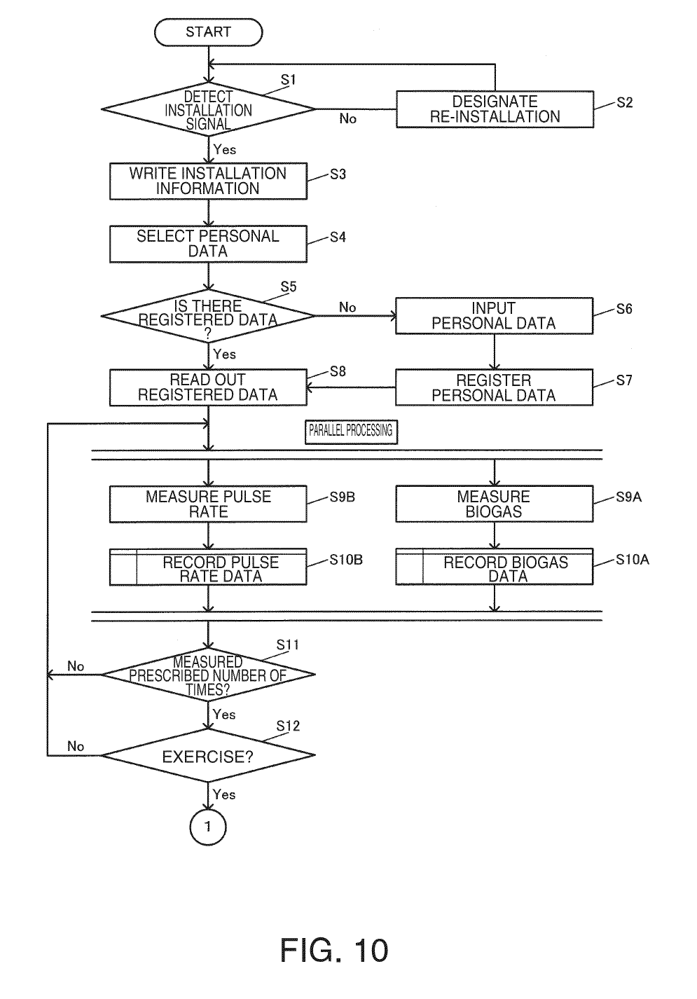

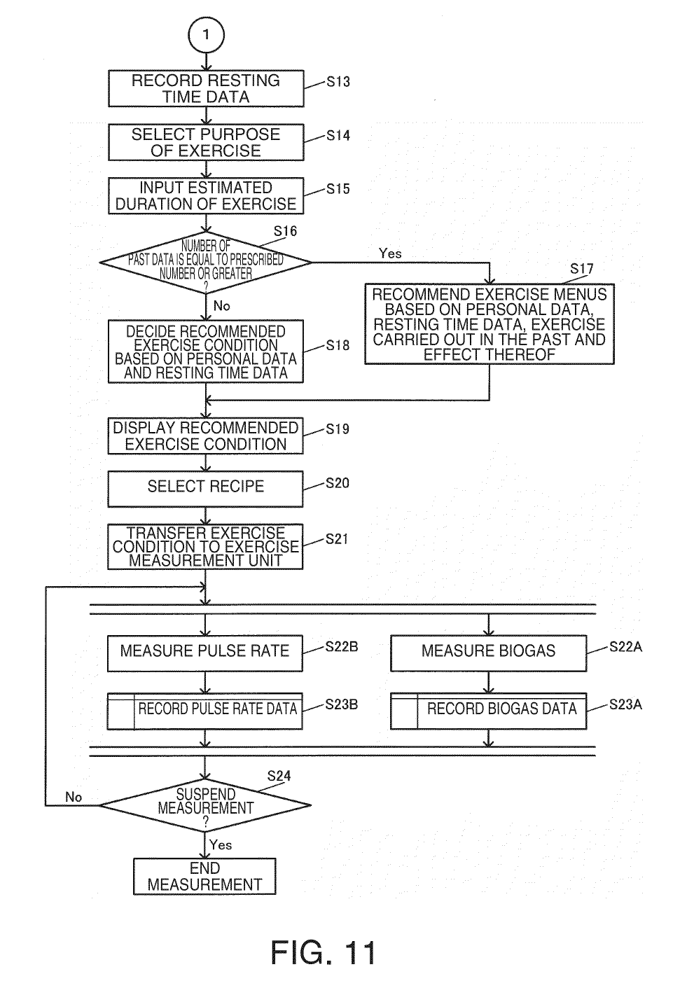

FIGS. 10 and 11 show an example of the flowchart of this embodiment. First, in FIG. 10, the installation of at least the acetone detection unit 3 and the ethanol detection unit 8 of the health management device 10 is confirmed via an installation signal (steps S1, S2). After that, when installation information of the installation signal is written (step S3), personal data of the subject is selected (step S4). If there is registered data, the data is read out (steps S5, S8). If there is no registered data, the personal data is inputted and registered and then read out (steps S5 to S8). The personal data includes name, age, height, body weight, current physical strength level, degree of diabetes and the like.

Next, in parallel processing, measurement of biogases (acetone, ethanol) and pulse rate is carried out at the time of resting and data thereof is recorded (steps S9A, S9B, S10A, S10B). This measurement is carried out a prescribed number of times (step S11). After the measurement at the time of resting is carried out a prescribed number of times, whether to start exercise or not is confirmed (step S12). Here, for example, if the decision to exercise is confirmed by an operation of an operation key (step S12), the key input may be used as an exercise start signal.

Subsequently, in FIG. 11, the data during the resting time implemented in steps S9A, S9B, S10A, S10B is recorded (step S13). Next, the purpose of exercise is inputted (step S14). The purpose of exercise may be insulin-sensitization, fat reduction or the like for a diabetic patient, and fat reduction, improvement in metabolic function or the like as measures against metabolic syndrome. Also, an estimated duration of exercise is inputted (step S15).

Next, whether the number of past data before the implementation of this time is a predetermined number or greater is confirmed (step S16). If the result of the determination is YES in step S16, the analysis unit 7 proposes the same exercise menu as in the past or the past exercise menu in which at least one of the exercise load and the duration of the exercise is revised, based on the personal data, the resting time data, the data before and after the exercise carried out in the past, and the result of analysis the data (step S17). The exercise menu of this type may be those described with reference to FIGS. 5 to 9.

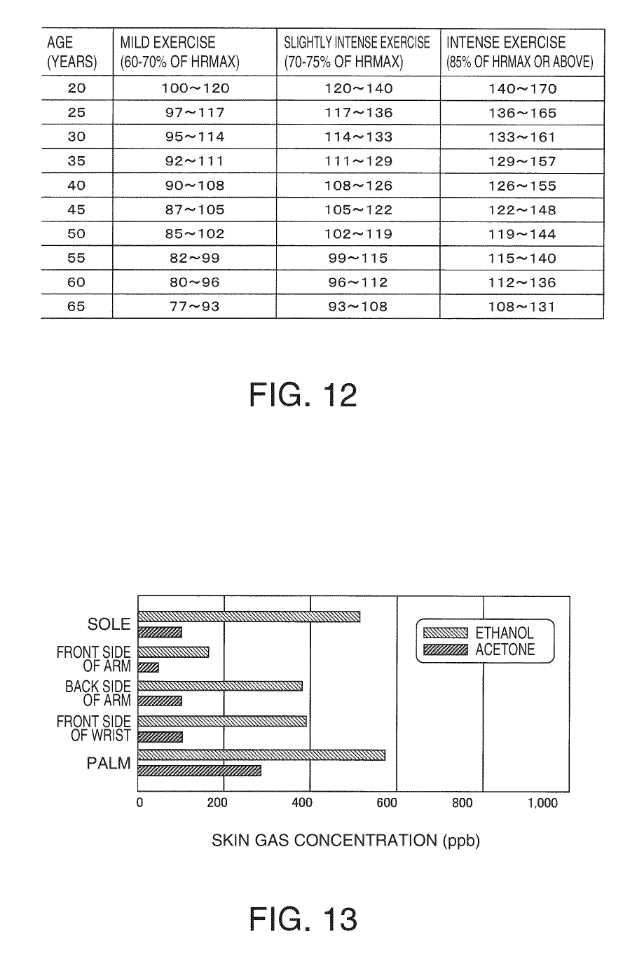

If the result of the determination in step S16 is NO, the analysis unit 7 proposes plural initial exercise menus, based on the personal data and the resting time data (step S18). The initial exercise menus can be, for example, three exercise menus corresponding to ages close to the real age of the subject, from among the exercises shown in FIG. 12. In the example of FIG. 12, one of the target heart rates HR is selected, and using the maximum heart rate (beats/minute) HRmax=220−age, the resting heart rate (beats/minute) HRrest, and the exercise intensity S(%)=(HRa−HRrest)/(HRmax−HRrest)×100, the initial menus are found based on the target heart rate HRtarget=(HRmax−HRrest)×S+HRrest.

The exercise menus recommended in step S18 or S19 are displayed on the display unit 6 (step S19), and for example, one of the plural exercise menus (recipes) is selected by the subject (step S20). This exercise menu may be transferred to an exercise measurement unit provided on an exercise machine as an exercising condition (step S21).

Subsequently, the exercise is started according to the preset exercise menu. During the exercise and after the suspension of the exercise, the measurement of biogas (acetone) and pulse rate is continued unless suspended, and data thereof is recorded (steps S22A, S22B, S23A, S23B, S24).

### 1.5. Biogas Detection Device

Next, a biogas detection device used as the acetone detection unit 3 or the ethanol detection unit 8 of the health management device 10 shown in FIG. 1 will be described. FIG. 13 shows an example of measurement of ethanol concentration and acetone concentration in the skin gas by site. The skin gas is gathered from each side for five minutes, using a silicone resin container, and ethanol and acetone concentrations are measured with a semiconductor gas chromatograph (SGEA-P2 by FIS Inc.) The acetone concentration in the breath gathered substantially at the same time is 5.4 ppm. As skin sites, five portions are selected and compared, that is, the sole (equivalent to the arch of the food), the front side and back side of the upper arm, the front side of the wrist (where a wristwatch is usually installed), and the palm. Although ethanol and acetone have different tendencies, it is found that both of the gases emanate well from the palm and that the palm is suitable as a biogas gathering portion. Therefore, a method of gathering the biogases from the palm is employed in this embodiment. Of course, the skin gas may be detected from other sites than the palm, and the breath may be detected.

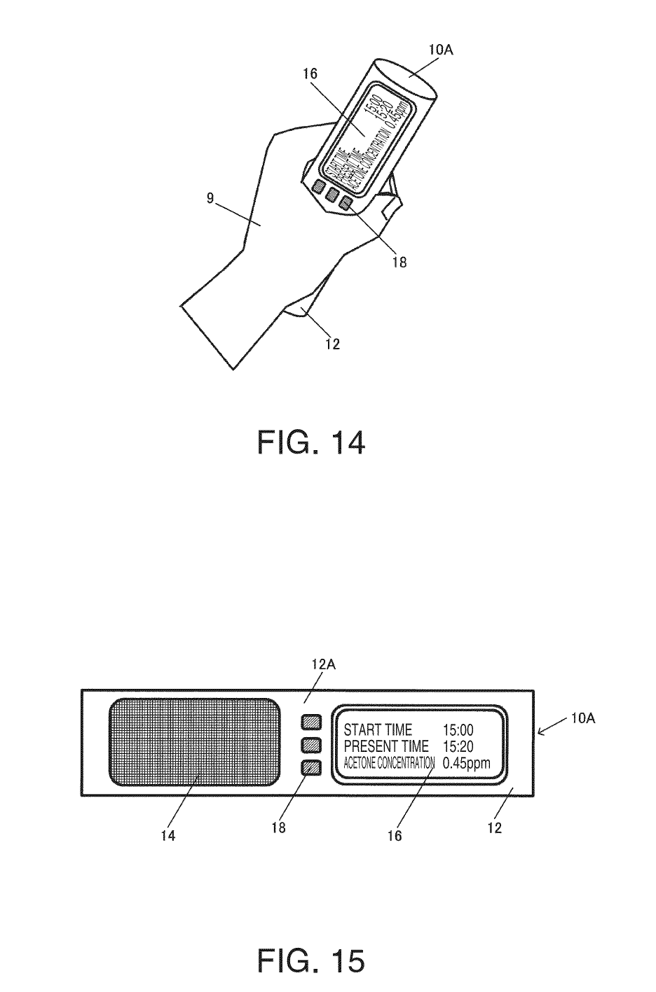

As shown in FIG. 13, considering that the concentration of the skin gas from the palm is higher than on the sole, the front side of the arm, the back side of the arm and the front side of the wrist, a skin gas detection device 10A held and carried by a hand 9 as shown in FIG. 14 is configured in this embodiment. The skin gas detection device 10A is a palm-held type that can carry out measurement easily with little physical restraint when the user walks or jogs on a treadmill or when the user walks or jogs outdoors.

The skin gas detection device 10A used as the acetone detection unit 3 or the ethanol detection unit 8 of the health management device 10 shown in FIG. 1 includes a sample gathering section 14, a display section 16 and an operation section 18, on a face side 12A of a casing 12, as shown in FIG. 15. As shown in FIG. 14, as the user holds a grip portion of the casing 12 with the left hand 9 covering the sampling gathering section 14, the user can operate the operation section 18 with the right hand and can see the display section 16. If the user holds the casing 12 with the right hand covering the sample gathering section 14, turning the display section 16 upside down can make it easier to see the display thereon.

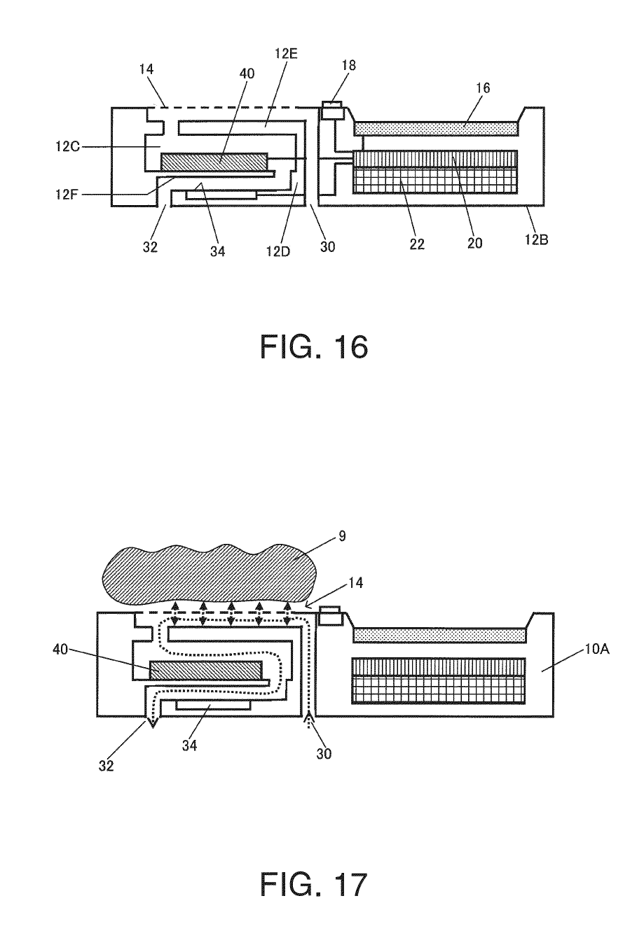

As shown in FIG. 16, inside the casing 12, a signal processing unit 20, a power supply unit 22 and the like are installed at a position facing the display section 16. The sample gathering section 14 provided on the casing 12 is made of, for example, mesh, and takes in the skin gas in contact with the palm. The inner space where the casing 12 faces the sample gathering section 14 is a hollow section 12C. The follow section 12C is divided by partitions 12D to 12F provided in the casing 12, thus forming a flow path.

One end of the flow path formed in the hollow section 12C communicates with an air intake portion 30 opening on a back side 12B. The other end of the flow path communicates with an air discharge portion 32 opening on the back side 12B. In the course of the flow path, for example, a detection unit 40 supported by the partition 12F is provided. In the direction of air flow from the air intake portion 30 toward the air discharge portion 32, a suction portion 34 is provided downstream from the detection unit 40.

The sample suction portion 34 is formed by a fan motor or tube pump or the like. In the case of a tube pump, as a rotary ring is rotated about an elastically deformable tube, the position where a rotary roller compresses the tube changes, thus discharging the gas to outside. When the sample suction portion 34 is driven, the pressure becomes negative on the side of the sample discharge portion 32 and the sample suction portion 34, causing the air entering the air intake portion 30 to pass through the flow path and become discharged from the air discharge portion 32, as shown in the path indicated by broken lines in FIG. 17. In this case, the mesh of the sample gathering section 14 communicates with the palm, and the skin gas emanated from the palm surface and entering through the mesh is caught in the air and introduced to the detection unit 40. Although the palm of the hand 9 is spaced apart from the sample gathering section 14 in FIG. 17, the palm and the sampling gathering section 14 are in tight contact with each other when the sample is gathered.

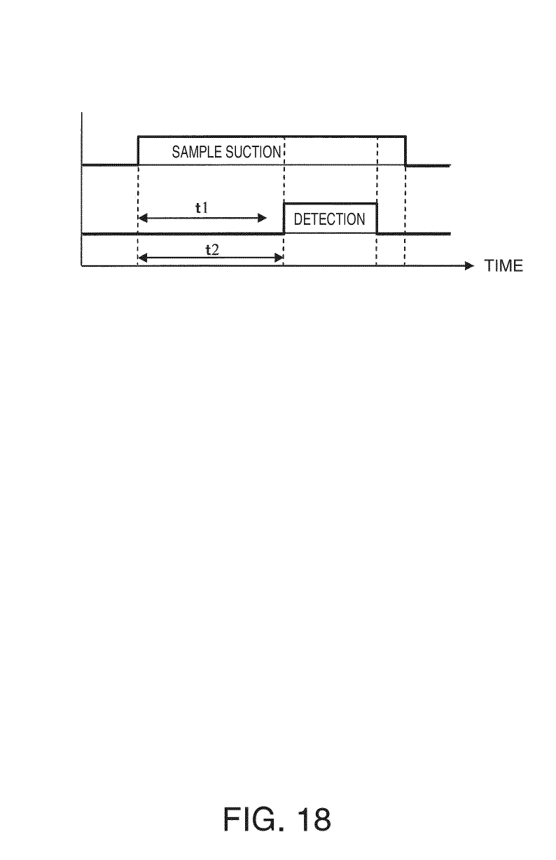

Skin gas components are detected at the detection unit 40. As shown in FIG. 18, if the air entering from the air intake portion 30 reaches the detection unit 40 after the lapse of a time t1 from the start of suction at the sample suction portion 34, the detection at the detection unit 40 is started after a time t2, which is slightly longer than the time t1. In the example of FIG. 18, the skin gas can be detected at the detection unit 40 while the skin gas is being sucked at the sample suction portion 34. After the detection is finished and then the skin gas is discharged, the sample suction portion 34 is stopped. Alternatively, after the suction, the driving of the sample suction portion 34 may be temporarily stopped and the skin gas may be detected then, or after the suction is stopped, the skin gas may be detected in an airtight state with a shutter closed, not shown. In such a case, when the detection of a target substance is finished, the skin gas is discharged from the sample suction portion 34 in order to discharge the skin gas actively.

Processing of a detection signal from the detection unit 40 and control of the sample suction portion 34 and the detection unit 40 are carried out by the signal processing control unit 20. Data of the detection result including the authentication data of the person, the date and time of exercise and the exercise condition are stored in a storage unit, not shown, and are displayed on the display section 16 according to need.

This skin gas detection device 10A is a portable type and therefore can use a primary battery or a secondary battery for the power supply unit 22. In the case of a primary battery, since there is an opening for battery replacement on the back side 12B of the casing 12, the battery can be replaced when battery depletion is shown in the display section 16. In the case of a secondary battery, since there is a connection portion on the back side 12B of the casing 12, a charger can be connected there to recharge the secondary battery when battery depletion is shown in the display section 16. As the end of charging is displayed, the charger can be removed and the device can be used again.

### 1.6. Detection Unit

### 1.6.1. Semiconductor Sensor

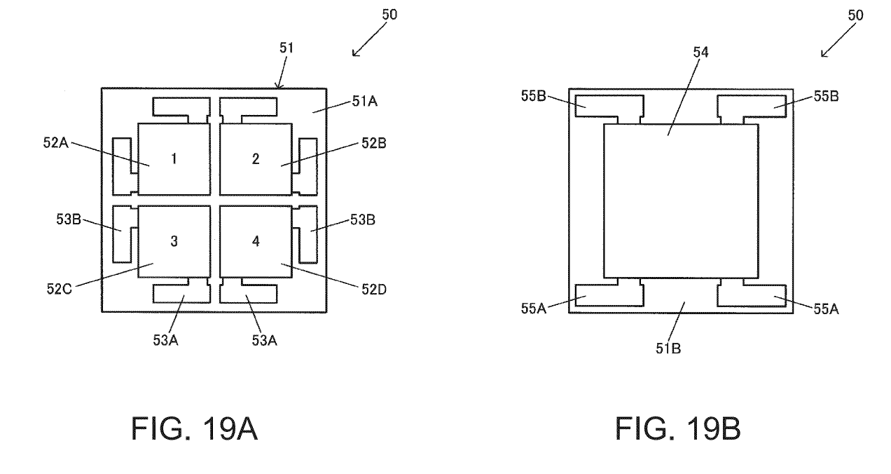

As the detection unit 40 shown in FIG. 16, a semiconductor sensor 50 shown in FIGS. 19A and 19B can be used. On a face side 51A of a substrate 51 of the semiconductor sensor 50, gas-sensitive material plates 52A to 52D and two electrodes 53A, 53B connected to each of gas-sensitive material plates 52A to 52D are provided, as shown in FIG. 19A. On a back side 51B of the substrate 51, a heater 54 and electrodes 55A, 55B connected thereto are provided, as shown in FIG. 19B. The gas-sensitive material plates 52A to 52D may be made of tin oxide (SnO₂), antimony-doped tin oxide (Sb:SnO₂), zinc oxide (ZnO), tungsten oxide (WO₃), indium tine oxide, titanium oxide (TiO₂), niobium-doped titanium oxide (Nb:TiO₂) or the like, and composite materials thereof and other additives may also be included.

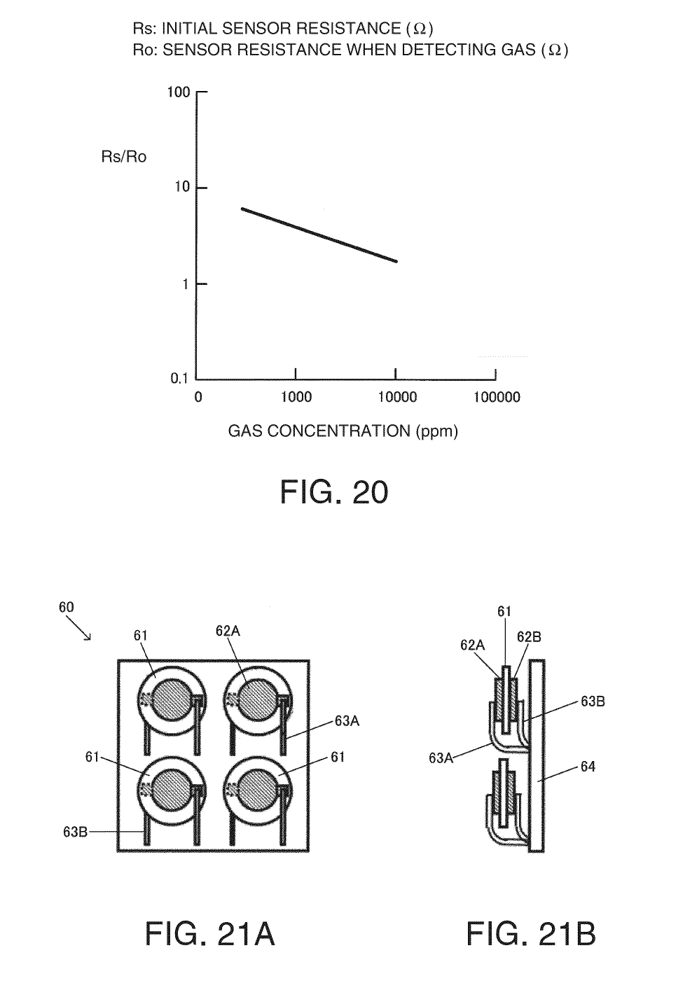

As a reducing gas is exposed to the gas-sensitive material plates 52A to 52D heated by the heater 54, oxygen adsorbed on the surfaces of the gas-sensitive material plates 52A to 52D is reduced and the potential barrier is lowered. Thus, electrons can move more easily and electric resistance falls. For example, the correlation between the concentration of ethanol contained in the skin gas and the sensor resistance ratio (Rs/R0) is shown in FIG. 20. Thus, the concentration of ethanol contained in the skin gas can be detected, based on the output from the semiconductor sensor 50.

### 1.6.2. Quartz Crystal Microbalance (QCM) Sensor

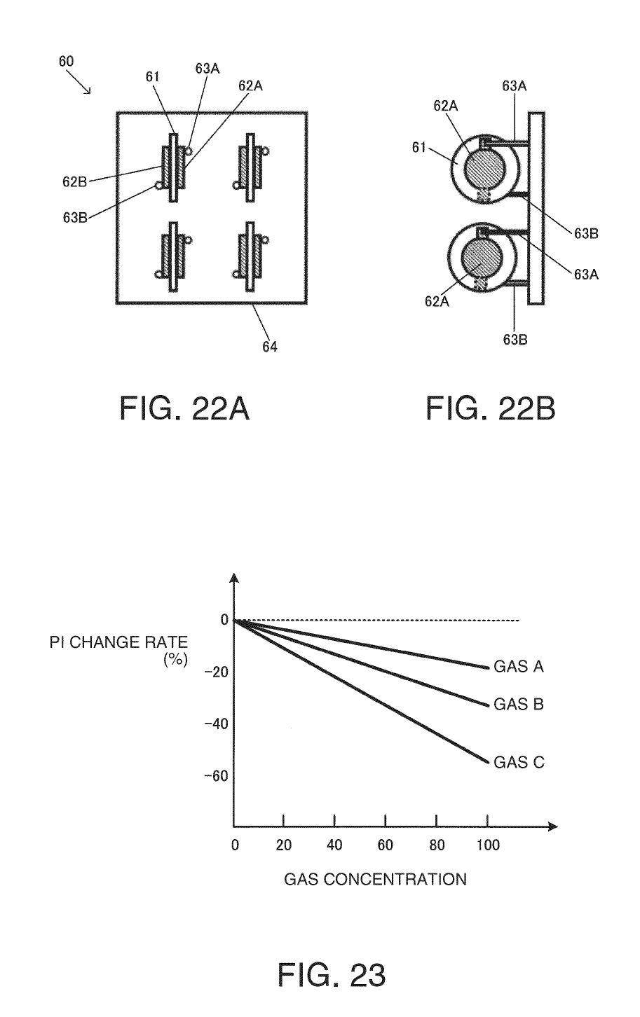

In a QCM sensor 60, electrodes 62A, 62B are arranged on both sides of a quartz crystal oscillator 61, and lead wires 63A, 63B connected to the electrodes 62A, 62B are fixed to a substrate 64, as shown in FIGS. 21A and 21B or FIGS. 22A and 22B. In FIG. 21B, the lead wires 63A, 63B are arranged parallel to the substrate 64, whereas in FIG. 22B, the lead wires 63A, 63B are arranged perpendicularly to the substrate 64. The configuration of FIGS. 22A and 22B has a better air flow, allowing the skin gas to contact both sides of the quartz crystal oscillator 61.

The frequency of the quartz crystal oscillator 61 changes according to the mass of the substance of the electrodes 62A, 62B. The relation between the amount of change in the frequency and the mass of the deposited substance can be expressed by the Sauerbrey equation. The frequency decreases as the mass of the deposited substance increases. The frequency increases as the mass of the deposited substance decreases. By preparing the calibration curves shown in FIG. 23 in advance, the concentration of the target substance can be found from the amount of change in the frequency.

### 1.6.3. SERS Sensor

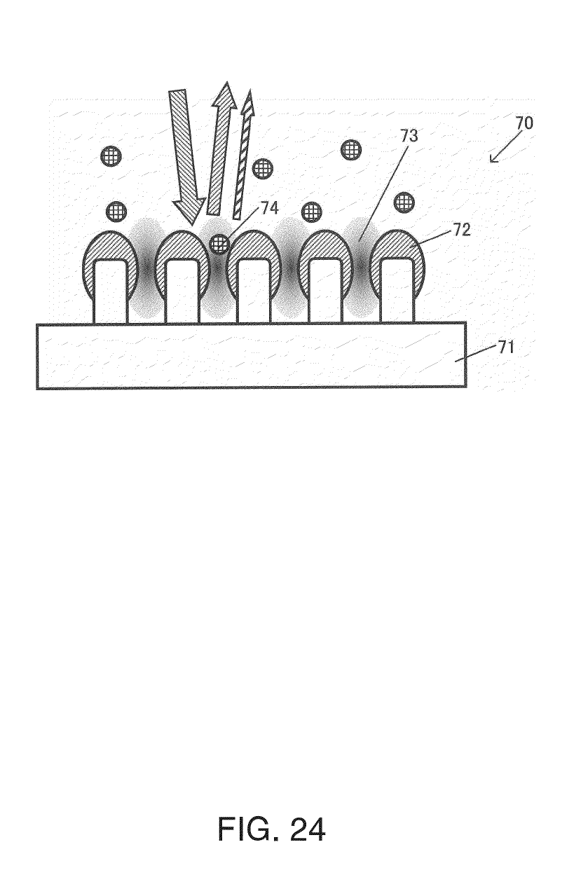

As shown in FIG. 24, in a SERS sensor 70, a sensor substrate 71 is arranged facing a flow path in which a gas is introduced, and excitation light (with the number of vibrations V) is cast onto the sensor substrate 71 from a light source. Most of the excitation light is scattered as Rayleigh-scattered light. The Rayleigh-scattered light has no change in the number of vibrations ν or wavelength from the incident light. A part of the excitation light is scattered as Raman-scattered light. The number of vibrations (ν−ν′ and ν+ν′) or wavelength of the Raman-scattered light reflects the number of vibrations ν′ of molecules (molecular vibrations). That is, the Raman-scattered light is a light reflecting the molecules of an inspection target. The molecular vibration energy may be added to the vibration energy or light energy of the Raman-scattered light. Such a shift in the number of vibrations (ν′) is called the Raman shift.

In the area where the incident light is cast onto metal nanoparticles 72 formed on the sensor substrate 71 shown in FIG. 24, an enhanced electric field 73 is formed between the neighboring metal nanoparticles 72. Particularly when the incident light is cast onto the metal nanoparticles 72 that are smaller than the wavelength of the incident light, the electric field of the incident light acts on free electrons existing on the surface of the metal nanoparticles 72 and causes resonance. Thus, an electric dipole is excited in the metal nanoparticles 72 by the free electrons, thereby forming the enhanced electric field 73 that is stronger than the electric field of the incident light. This is also called the localized surface plasmon resonance (LSPR). This phenomenon is unique to electric conductors such as metal nanoparticles 72 or the like having a size of 1 to 500 nm that is smaller than the wavelength of the incident light.

In FIG. 24, surface enhanced Raman scattering (SERS) occurs when the incident light is cast onto the sensor substrate 71. That is, as a target molecule 74 enters the enhanced electric field 73, the Raman-scattered light generated by the target molecule 74 is enhanced in the enhanced electric field 73 and the signal intensity of the Raman-scattered light increases. In such surface enhanced Raman scattering, detection sensitivity can be raised even if the amount of the target molecule 74 is very small.

### 2. Second Embodiment

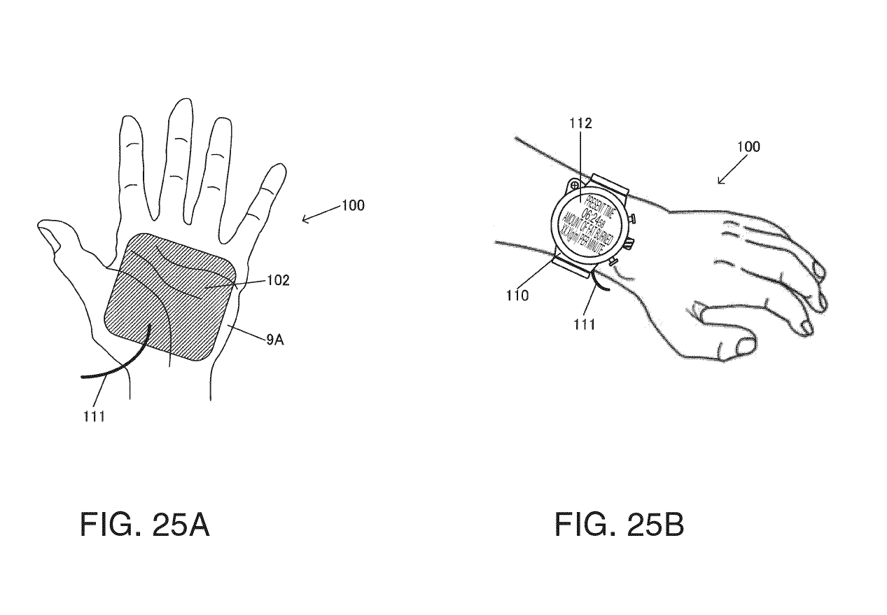

Different from the first embodiment in which the casing 12 is configured as the palm-held type, a skin gas detection device 100 according to a second embodiment shown in FIGS. 25A and 25B has a pad main body 102 as a casing that is joined, for example, to a palm 9A of a left hand 9. In the skin gas detection device 100, a device main body unit 110 connected to the pad main body 102 via a cable 111 is in the form of, for example, a wristwatch, and installed on the wrist of the left hand 9, and the pad main body 102 is made thin, thus securing the operability on the palm 9A. The device main body 110 is provided with a display unit 112, a signal processing unit (not shown) and the like.

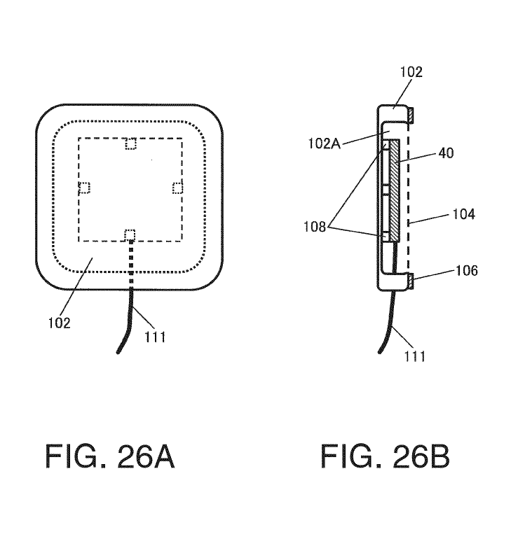

As shown in FIGS. 26A and 26B, the pad main body 102 has a mesh-like sample gathering section 104 on a surface contacting the palm 9A. The sample gathering section 104 can take the skin gas into a hollow portion 102A of the pad main body 102 through the surface where the pad main body 102 contacts the palm 9A. To cause the sample gathering section 104 to contact the palm, it is effective to join the pad main body 102 to the palm 9A as shown in FIG. 25A, other than holding the pad main body as shown in FIG. 14. The pad main body 102 can be joined to the palm 9A by means of an adhesive sheet 106, for example. A detection unit 40 is fixed to the pad main body 102 via a support 108. It should be noted that the air intake portion 30, the air discharge portion 32 and the sample suction portion 34 described with reference to FIG. 16 can also be applied to the pad main body 102.

### 3. Third Embodiment

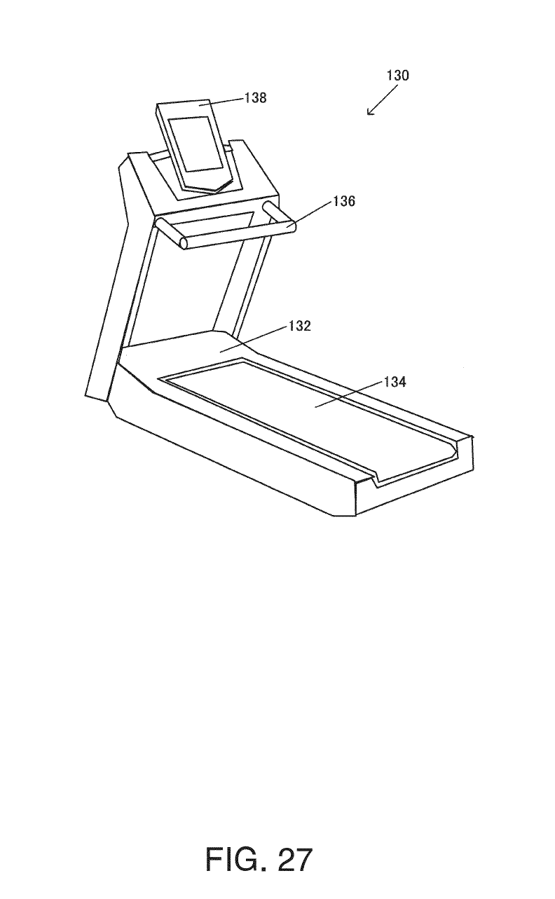

FIG. 27 shows an exercise machine (health management device) 130 in which a skin gas detection device 120 according to a third embodiment of the invention is incorporated. The exercise machine 130 can include the various control system blocks shown in FIG. 1. The exercise machine (health management device) 130 is a treadmill in which the user walks or jogs on a belt 134 that travels relative to a casing 132. The treadmill 130 has a handle (holding portion) 136 as a part of the casing 132. The casing 132 is provided with a display unit 138. The treadmill 130 is an exercise machine that has a function of gathering the skin gas emanated from the human skin surface and detects components such as acetone in the skin gas.

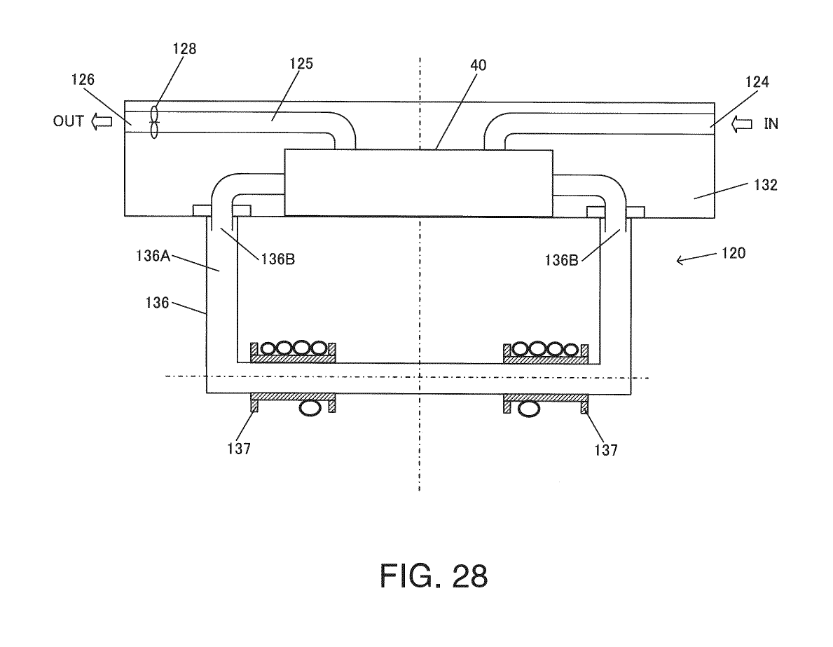

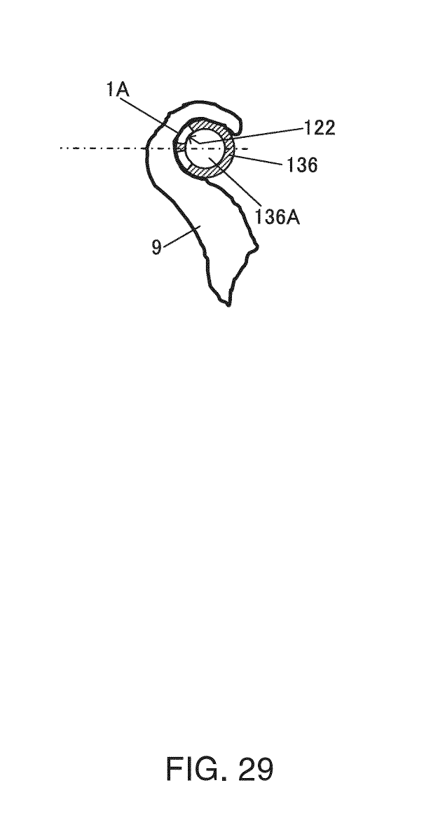

FIGS. 28 and 29 show the skin gas detection device 120. In grip portions 137 for both hands of the handle 136 shown in FIG. 28, a sample gathering section 122 having an opening on a surface contacting the palm 9A is provided, as shown in FIG. 29. Since the handle 136 is hollow, the skin gas enters a hollow portion 136A via the sample gathering section 122.

The casing 132 of the treadmill 130 is provided with a flow path 125 faced with the detection unit 40. One end of the flow path 125 is an air intake portion 124. A suction portion 128 is provided near an air discharge portion 126 at the other end. The hollow portion 136A of the handle 136 communicates with the flow path 125 via a communication port 136B. Therefore, when an air flow is generated in the flow path 125, the skin gas in the hollow portion 136A of the handle 136 is sucked toward the flow path 125 and contacts the detection unit 40.

The biogas emanated from the body (in this example, the palm) enters the handle portion of the treadmill. The biogas entering the handle portion is introduced into the biogas detection unit arranged inside the main body unit of the treadmill.

A person skilled in the art can readily understand that a number of modifications can be made without substantially departing from the novel elements and effects of the invention. Therefore, all such modifications should be included in the scope of the invention. For example, a term that is described together with a different term that has a broader meaning or the same meaning at least once in the specification or drawings can be replaced with that different term anywhere in the specification or drawings. Also, the configurations and operations of the health management device 10 and the like are not limited to those described in the embodiments and various modifications can be made.

The entire disclosure of Japanese Patent Application No. 2013-068061, filed Mar. 28, 2013 is expressly incorporated by reference herein.

What is claimed is:

1. A health management device comprising:
a signal output unit which outputs a start signal indicating a start timing when application of an exercise load on a subject is started;
an acetone detection unit which detects an acetone concentration in a biogas of the subject;
a clock unit which measures time;
a storage unit which stores the start signal and the acetone concentration outputted from the acetone detection unit together with the time; and
a display unit which displays a first transition data group of the acetone concentration outputted from the acetone detection unit before the start signal is received, and a second transition data group of the acetone concentration outputted from the acetone detection unit after the start signal is received, based on an output from the storage unit.

2. The health management device according to claim 1, wherein the signal output further outputs an end signal indicating an end timing when the application of the exercise load on the subject is ended,
the storage unit further stores the end signal and the acetone concentration outputted from the acetone detection unit, and
the display unit displays a third transition data group of the acetone concentration outputted from the acetone detection unit after the end signal is received, together with the first transition data group and the second transition data group.

3. The health management device according to claim 2, further comprising an analysis unit which analyzes a metabolic state, based on at least one of the first transition data group, the second transition data group and the third transition data group stored in the storage unit,
wherein the display unit displays a result of analysis from the analysis unit.

4. The health management device according to claim 3, further comprising a recording unit which records meal time,
wherein the analysis unit analyzes the metabolic state including a correlation with the meal time, based on at least one of the first transition data group, the second transition data group and the third transition data group stored in the storage unit.

5. The health management device according to claim 1, wherein the signal output unit is a biological signal detection unit which detects a biological signal that changes according to the magnitude of the exercise load on the subject, and
the biological signal is stored in the storage unit, and the display unit displays the biological signal.

6. The health management device according to claim 5, wherein the biological signal detection unit is a pulse rate detection unit which detects a pulse rate of the subject.

7. The health management device according to claim 3, wherein the recording unit is an ethanol detection unit which detects an ethanol concentration in the biogas of the subject.

8. The health management device according to claim 3, wherein data groups about the same subject are accumulated repeatedly in the storage unit,
the analysis unit analyzes an exercise level that is recommended with respect to at least one of the exercise intensity and the duration of the exercise, based on the data groups accumulated in the storage unit, and
the display unit displays the recommended exercise level from the analysis unit.

9. The health management device according to claim 8, wherein if the amount of the data groups that are accumulated is small, the analysis unit recommends an exercise menu selected from plural initial exercise menus in which at least one of the exercise intensity and the duration of the exercise varies, based on the data groups.
